(19) United States
(12) Patent Application Publication
(10) Pub. No.: US 2024/0393031 A1
(43) Pub. Date: Nov. 28, 2024

(54) NECK HANGING TEMPERATURE REGULATION DEVICE

(71) Applicant: Shenzhen Replay Technology Co., Ltd., Shenzhen (CN)

(72) Inventor: LIFANG CHEN, Shenzhen (CN)

(21) Appl. No.: 18/535,905

(22) Filed: Dec. 11, 2023

(30) Foreign Application Priority Data
May 25, 2023 (CN) 202321285898.2

(51) Int. Cl. F25D 17/06

(52) U.S. Cl. CPC F25D 17/06

(57) ABSTRACT

The present disclosure discloses a neck hanging temperature regulation device including a first bracket, a connection part, and two second brackets. The first bracket is provided with a refrigeration component and two first blowing components located on two sides of the refrigeration component. The first blowing component includes a first inner shell component and a first fan, and the first fan is connected to a first air inlet and a first air outlet, the first inner shell component is provided with an air inlet window, which is connected to the first air inlet. Heat emitted by the refrigeration component can enter through the air inlet window and disperse through the first air outlet; by connecting the refrigeration component with the air inlet window, the heat emitted by the refrigeration component enters through the air inlet window and dissipates through the first air outlet.

### CROSS-REFERENCE TO RELATED APPLICATIONS

This application claims priority to Chinese Patent Application No. 202321285898.2, filed on May 25, 2023, which is hereby incorporated by reference in its entirety.

### TECHNICAL FIELD

The present disclosure relates to the field of portable temperature control device technologies, and in particular, to a neck hanging temperature regulation device.

### BACKGROUND

With the improvement of living standards, people have also put forward higher requirements for the comfort and convenience of life. In recent years, various portable fans have emerged on the market, such as handheld fans, neck mounted fans, etc., providing users with cool air anytime and anywhere. Compared to handheld fans, which require users to hold the fan in their hands, a neck mounted fan can free up users' hands and is more popular among the public, especially in sports, outdoor activities, and other occasions, greatly facilitating users.

In order to achieve a cooling function of the neck hanging fan on the market, a refrigeration component is added to the neck hanging fan to achieve a purpose of temperature regulation. During use, the refrigeration component will continue to dissipate heat. Although a neck hanging fan bracket has a heat dissipation port, the heat dissipation speed is relatively slow and the efficiency is low, which causes a large amount of heat around the neck, affects the user's use and uncomfortable experience; and usually the neck hanging fan is cooled in a middle, with air blowing on two sides. An air output and range are relatively small, and the air volume is not large.

### SUMMARY

To address the shortcomings in existing technology, the present disclosure provides a neck hanging temperature regulation device, which has characteristics of increasing air output and accelerating heat dissipation.

The present disclosure adopts the following technical solutions to implement.

A neck hanging temperature regulation device can be worn on a human neck, and includes a first bracket, a connection part, and two second brackets. The two second brackets are respectively connected to two ends of the first bracket through the connection part. The second bracket is provided with a second blowing component, and the first bracket is provided with a refrigeration component, and two first blowing components located on two sides of the refrigeration component. The first bracket is provided with a first air inlet and a first air outlet corresponding to the first blowing component, and a second air inlet corresponding to the refrigeration component. Each first blowing component includes a first inner shell component and a first fan, and the first inner shell component forms an accommodation chamber in an internal space of the first bracket, the first fan is accommodated in the accommodation chamber. The first fan is connected to the first air inlet and the first air outlet; the first inner shell component is provided with an air inlet window. The air inlet window is connected to the first air inlet; heat emitted by the refrigeration component can enter through the air inlet window and dissipate through the first air outlet.

In an implementation mode of the present disclosure, the first blowing component is a single-sided air inlet, and the first air inlet is provided on a side of the first bracket away from the neck.

In an implementation mode of the present disclosure, the first inner shell component includes two partitions, which are located on two sides of the first fan; a first air duct is formed between the two partitions and the first fan, and an extension direction of the first air duct is perpendicular to a length direction of the first bracket.

In an implementation mode of the present disclosure, a middle part of one partition is bent outward and adapted to a circumferential contour of the first fan.

In an implementation mode of the present disclosure, the refrigeration component includes a cooling fan, a cooling element, a heat dissipation element, and a cool guide plate. Two ends of the cooling element are respectively connected to the heat dissipation element and the cool guide plate, and the cooling fan is connected to the heat dissipation element. The cool guide plate is provided near the human neck, and the second air inlet corresponds to the cooling fan.

In an implementation mode of the present disclosure, a heat dissipation channel is formed between the heat dissipation element and the air inlet window; an air inlet channel is formed between the first air inlet and the first fan, and the heat dissipation channel and the air inlet channel are at least partially overlap.

In an implementation mode of the present disclosure, the second bracket is provided with a battery, a control board, and a control switch; the second blowing component includes a second fan, a second inner shell component; and the battery, the control board, the control switch, the first fan, and the second fan are electrically connected to each other.

In an implementation mode of the present disclosure, an end of the second inner shell component is adapted to the second fan, and the other end thereof extends along a length direction of the second bracket.

In an implementation mode of the present disclosure, the second bracket is provided with a third air inlet and a third air outlet corresponding to the second fan, and a second air duct is formed between the second fan and the third air outlet. An area of the second air duct gradually decreases from a direction close to the second fan to away from the second fan.

In an implementation mode of the present disclosure, the connection part includes a first connector, a second connector, and an intermediate connector; the first connector is fixedly connected to the second bracket, the second connector is fixedly connected to the first bracket; and two ends of the intermediate connector are respectively sleeved with the first connector and the second connector.

In an implementation mode of the present disclosure, the intermediate connector is a hollow silicone member, and the first and second connectors are made of hard material.

Compared with existing technologies, the beneficial effects of the present disclosure are:
1. a first blowing component is provided in the first bracket, a second blowing component is provided in the second bracket, and multiple blowing components are provided to increase an air output of the neck hanging fan and expand an air output range;
2. the first bracket is provided with a refrigeration component, a first blowing component is provided on two sides of the refrigeration component, the refrigeration component is connected to an air inlet window, and heat emitted by the refrigeration component enters through an air inlet window and disperses through the first air outlet; through this arrangement, heat dissipation of the refrigeration component is accelerated, thereby rendering a user experience more comfortable.

### BRIEF DESCRIPTION OF DRAWINGS

In order to provide a clearer explanation of the technical solution of the embodiments of the present disclosure, a brief introduction will be made to the drawings required in the description of the embodiments. It is evident that the drawings in the following description are some embodiments of the present disclosure. For those skilled in the art, other drawings can be obtained based on these drawings without creative work.

FIG. 1 is a schematic diagram of a structure of a neck hanging temperature regulation device of the present disclosure.

FIG. 2 is a schematic diagram of an internal structure of a second bracket in FIG. 1.

FIG. 3 is a schematic diagram of an internal structure of a first bracket in FIG. 1.

FIG. 4 is a schematic diagram of an internal structure of a refrigeration component in FIG. 1.

FIG. 5 is a schematic diagram of a structure of a first blowing component in FIG. 1.

### DESCRIPTION OF EMBODIMENTS

In order to facilitate a clear understanding of the technical means, technical features, objectives, and effects achieved by the present disclosure, the following will provide a clear and complete description of the technical solution in the embodiments of the present disclosure, in combination with the drawings. Obviously, the described embodiments are only a part of the embodiments of the present disclosure, not all of them.

In the description of the present disclosure, it should be noted that terms “up”, “down”, “inside”, “outside”, etc. indicate a direction or position relationship based on the direction or position relationship shown in the drawings, only for the convenience of describing the present disclosure and simplifying the description, rather than indicating or implying that the device or component referred to must have a specific orientation, be constructed and operated in a specific orientation. Therefore, it cannot be understood as a limitation on the present disclosure. In addition, terms “first” and “second” are only used to describe the purpose and cannot be understood as indicating or implying relative importance.

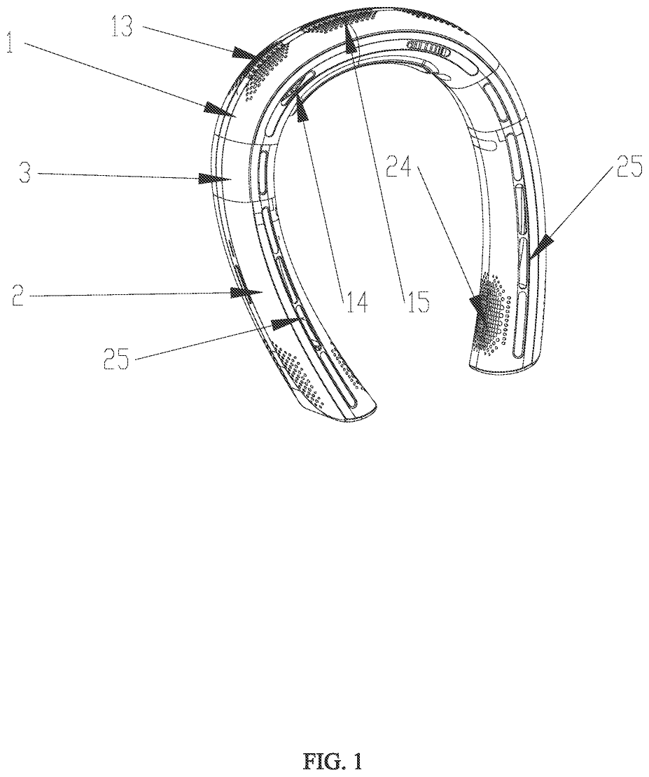

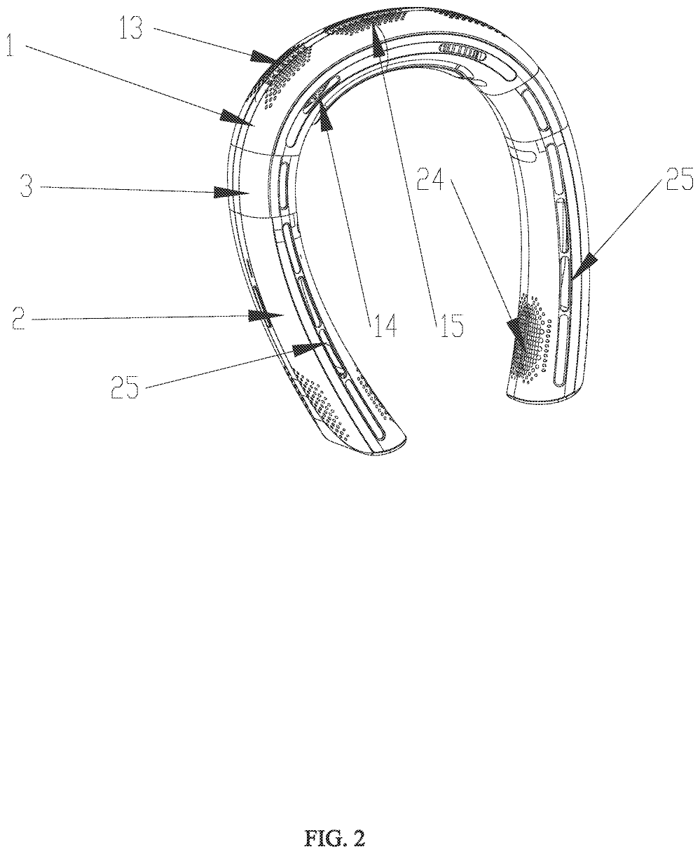

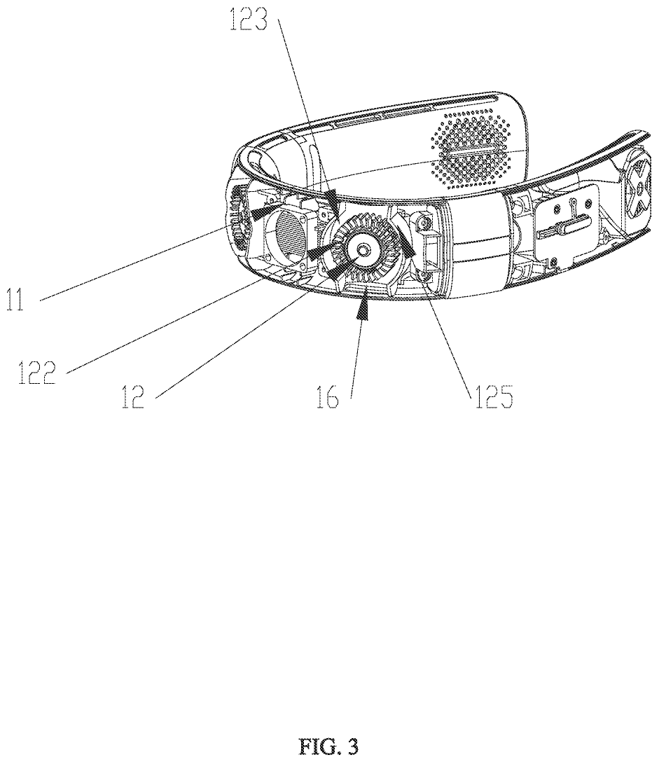

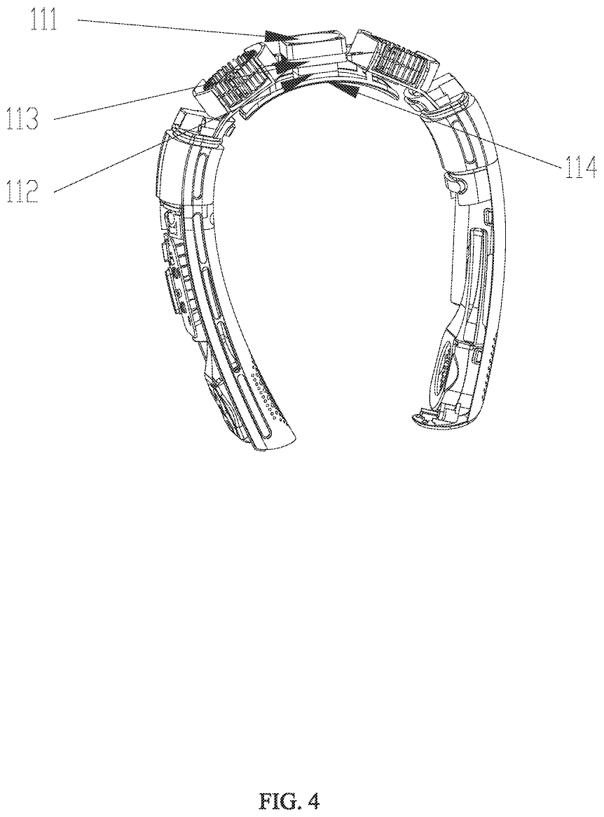

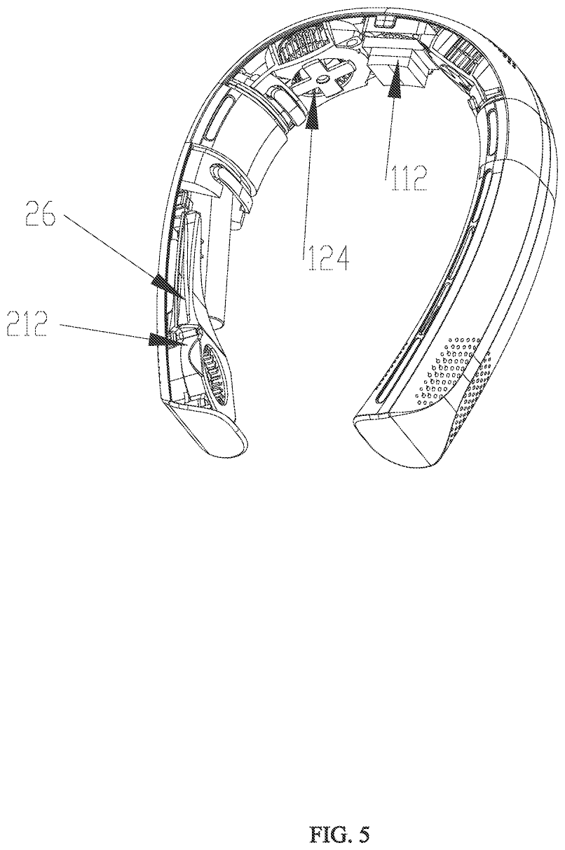

As shown in FIGS. 1-5, a neck hanging temperature regulation device of the present disclosure can be worn on a human neck, which includes a first bracket 1, a connection part 3, and two second brackets 2. The wo second brackets 2 are respectively connected to two ends of the first bracket 1 through the connection part 3. The second bracket 2 is provided with a second blowing component 21, and the first bracket 1 is provided with a refrigeration component 11 and two first blowing components 12, the two first blowing components 12 are located on left and right sides of the refrigeration component 11, respectively. The first bracket 1 is provided with a first air inlet 13 and a first air outlet 14 corresponding to the first blowing component 12, and a second air inlet 15 corresponding to refrigeration component 11. The first blowing component 12 includes a first inner shell component 121 and a first fan 122. The first inner shell component 121 forms an accommodation chamber 123 in an internal space of the first bracket 1, and the first fan 122 is accommodated in the accommodation chamber 123, the first fan 122 is connected to the first air inlet 13 and the first air outlet 14. The first inner shell component 121 is further provided with an air inlet window 124, which is connected to the first air inlet 13. Air enters from the first air inlet 13 and blows out from the first air outlet 14 after passing through the first fan 122. Because the refrigeration component 11 and the first blowing component 12 are both distributed in the internal space of the first bracket 1, the refrigeration component 11 and the air inlet window 124 are connected. Therefore, heat emitted by the refrigeration component 11 during operation can enter through the air inlet window 124 and dissipate through the first air outlet 14.

Specifically, the first blowing component 12 is a single-sided air inlet, and the first air inlet 13 is provided on a side of the first bracket 1 away from the neck.

Specifically, the heat emitted by refrigeration component 11 is sucked in by the first fan 122 from the air inlet window 124, because when the first fan 122 is working, it will suck the air at the first air inlet 13 into the air inlet window 124. When the heat dissipated by the refrigeration component 11 flows near the air inlet window 124, it will also be sucked in. Therefore, the first blowing components 12 are installed on two sides of refrigeration component 11, and the first fans 122 on two sides can suck in and blow out the heat emitted by the refrigeration component 11 while blowing, thereby achieving a goal of accelerating heat dissipation.

Specifically, although the heat emitted by the refrigeration component 11 and a natural wind blown out by the first blowing component 12 are both blown out from the first air outlet 14, the temperature of the air outlet does not increase significantly. Compared with the air flow of the air outlet, a proportion of heat dissipation is very small, and the temperature of the air outlet is hardly changed, so it will not affect a user's blowing experience.

In an implementation mode, the first inner shell component 121 includes two partitions 125, which are located on two sides of the first fan 122. A first air duct 16 is formed between the two partitions 125 and the first fan 122, and an extension direction of the first air duct 16 is perpendicular to a length direction of the first bracket 1.

Specifically, the partition 125 is adjacent to the left and right sides of the first fan 122, allowing the first fan 122 to directly exhaust from top and bottom sides, rendering the exhaust more concentrated.

In an implementation mode, a middle part of one partition 125 is bent outward and adapted to a circumferential contour of the first fan 122.

In an implementation mode, the refrigeration component 11 includes a cooling fan 111, a cooling element 112, a heat dissipation element 113, and a cool guide plate 114. Two ends of the cooling element 112 are respectively connected to the heat dissipation element 113 and the cool guide plate 114, and the cooling fan 111 is connected to the heat dissipation element 113. The cool guide plate 114 is provided near the human neck, and the second air inlet 15 corresponds to the cooling fan 111.

Specifically, the cooling element 112 is a semiconductor cooling block, the cool guide plate 114 is thermally conductive to a cooling surface of a semiconductor cooling block, and the heat dissipation element 113 is thermally conductive to the cooling surface of the semiconductor cooling block. After the semiconductor cooling block is powered on, the temperature on one side decreases and the temperature on the other side increases. Therefore, one side is the cooling surface, and the other side is the heating surface. The temperature near the human neck decreases. The cooling capacity of the cooling surface of the semiconductor cooling block is transmitted through the cool guide plate 114 to cool the human body. The built-in heat dissipation element 113 and the cooling fan 111 are used to dissipate heat from the heating surface of the semiconductor cooling block, thereby achieving heat dissipation function.

Specifically, during an operation, the cooling element 112 inevitably heats up and generates a certain amount of heat. If the heat is not processed, it will greatly reduce the cooling effect of the cooling element 112. Therefore, it is necessary to use the heat dissipation element 113 to improve the aforementioned problem. There is a temperature difference between cooling element 112 and heat dissipation element 113, so the heat of the cooling element 112 is transferred to the heat dissipation element 113 through heat conduction connection, and then the heat of a radiator is blown away by the cooling fan 111, which does not affect the cooling effect of the cooling element 112; a thermal conduction connection between the cool guide plate 114 and the cooling element 112 refers to the temperature difference between the cooling element 112 and the cool guide plate 114 during refrigeration operation. The cooling element 112 transfers the cooling amount to the cool guide plate 114, allowing the cool guide plate to come into contact with the human neck to achieve refrigeration conduction.

In an implementation mode, a heat dissipation channel is formed between the heat dissipation element 113 and the air inlet window 124, and an air inlet channel is formed between the first air inlet 13 and the first fan 122. The heat dissipation channel and the air inlet channel are at least partially overlapped.

Specifically, the heat dissipation element 113 and the cooling fan 111 are used to dissipate heat from the cooling element 112. In an existing technology, heat dissipation is relatively simple and directly disperses from a heat dissipation port, resulting in lower efficiency of heat dissipation. Therefore, the heat dissipation channel is arranged to partially overlap with the air inlet channel, that is, the heat dissipation channel passes through the air inlet window 124, and the air inlet channel passes through the air inlet window 124. Natural wind and heat are both sucked in near a position of the air inlet window 124, which accelerates an efficiency of heat dissipation.

In an implementation mode, the second bracket 2 is provided with a battery 27, a control board 22, and a control switch 23. The second blowing component 21 includes a second fan 211 and a second inner shell component 212. The battery 27, the control board 22, the control switch 23, the first fan 122, and the second fan 211 are electrically connected to each other.

In an implementation mode, an end of the second inner shell component 212 is adapted to the second fan 211, and the other end thereof extends along a length direction of the second bracket 2.

In an implementation mode, the second bracket 2 is provided with a third air inlet 24 and a third air outlet 25 corresponding to the second fan 211. A second air duct 26 is formed between the second fan 211 and the third air outlet 25. An area of the second air duct 26 gradually decreases from a direction close to the second fan 211 to away from the second fan 211.

In an implementation mode, the connection part 3 includes a first connector 31, a second connector 32, and an intermediate connector 33. The first connector 31 is fixedly connected to the second bracket 2, the second connector 32 is fixedly connected to the first bracket 1, and two ends of the intermediate connector 33 are respectively sleeved with the first connector 31 and the second connector 32.

In an implementation mode, the intermediate connector 33 is a hollow silicone member, and the first connector 31 and the second connector 32 are made of hard material.

The above shows and describes the basic principles, main features, and advantages of the present disclosure. Technicians in this filed should understand that the present disclosure is not limited by the above embodiments. The above embodiments and instructions only explain the principle of the present disclosure. Without departing from the spirit and scope of the present disclosure, there will be various changes and improvements in the present disclosure, all of which fall within the protection scope of the present disclosure. The protection scope of the present disclosure is defined by the claims and their equivalents.

What is claimed is:

1. A neck hanging temperature regulation device, which can be worn on a human neck, and comprises a first bracket, a connection part, and two second brackets; the two second brackets are respectively connected to two ends of the first bracket through the connection part;
wherein the second bracket is provided with a second blowing component, and the first bracket is provided with a refrigeration component and two first blowing components located on two sides of the refrigeration component, the first bracket is provided with a first air inlet and a first air outlet corresponding to the first blowing components, and a second air inlet corresponding to the refrigeration component; each first blowing component comprises a first inner shell component and a first fan, and the first inner shell component forms an accommodation chamber in an internal space of the first bracket; the first fan is accommodated in the accommodation chamber, and the first fan is connected to the first air inlet and the first air outlet; the first inner shell component is provided with an air inlet window, which is connected to the first air inlet; heat emitted by the refrigeration component can enter through the air inlet window and disperse through the first air outlet;
the first air inlet is provided on a side of the first bracket away from the neck.

2. The neck hanging temperature regulation device according to claim 1, wherein the first inner shell component comprises two partitions, which are located on two sides of the first fan; a first air duct is formed between the two partitions and the first fan, and an extension direction of the first air duct is perpendicular to a length direction of the first bracket.

3. The neck hanging temperature regulation device according to claim 2, wherein a middle part of one partition is bent outward and adapted to a circumferential contour of the first fan.

4. The neck hanging temperature regulation device according to claim 1, wherein the refrigeration component comprises a cooling fan, a cooling element, a heat dissipation element, and a cool guide plate; two ends of the cooling element are respectively connected to the heat dissipation element and the cool guide plate, and the cooling fan is connected to the heat dissipation element; the cool guide plate is provided near the human neck, and the second air inlet corresponds to the cooling fan.

5. The neck hanging temperature regulation device according to claim 4, wherein a heat dissipation channel is formed between the heat dissipation element and the air inlet window, an air inlet channel is formed between the first air inlet and the first fan, and the heat dissipation channel and the air inlet channel are at least partially overlapped.

6. The neck hanging temperature regulation device according to claim 1, wherein the second bracket is provided with a battery, a control board, and a control switch; the second blowing component comprises a second fan, a second inner shell component; and the battery, the control board, the control switch, the first fan, and the second fan are electrically connected to each other.

7. The neck hanging temperature regulation device according to claim 6, wherein an end of the second inner shell component is adapted to the second fan, and the other end thereof extends along a length direction of the second bracket.

8. The neck hanging temperature regulation device according to claim 7, wherein the second bracket is provided with a third air inlet and a third air outlet corresponding to the second fan, and a second air duct is formed between the second fan and the third air outlet; an area of the second air duct gradually decreases from a direction close to the second fan to away from the second fan.

9. The neck hanging temperature regulation device according to claim 1, wherein the connection part comprises a first connector, a second connector, and an intermediate connector; the first connector is fixedly connected to the second bracket, the second connector is fixedly connected to the first bracket, and two ends of the intermediate connector are respectively sleeved with the first connector and the second connector.

10. The neck hanging temperature regulation device according to claim 9, wherein the intermediate connector is a hollow silicone member, the first and second connectors are made of hard material.
